(19) United States
(12) Patent Application Publication
(10) Pub. No.: US 2026/0009016 A1
(43) Pub. Date: Jan. 8, 2026

(54) METHOD AND DEVICE FOR NUCLEIC ACID PURIFICATION

(71) Applicant: The Hong Kong University of Science and Technology, Hong Kong (CN)

(72) Inventors: Levent YOBAS, Hong Kong (CN); Yang BU, Hong Kong (CN); Sheng NI, Hong Kong (CN)

(21) Appl. No.: 19/329,683

(22) Filed: Sep. 16, 2025

Related U.S. Application Data
(63) Continuation-in-part of application No. PCT/IB2024/000192, filed on Apr. 18, 2024.
(60) Provisional application No. 63/496,956, filed on Apr. 19, 2023.

(51) Int. Cl. C12N 15/10

(52) U.S. Cl. CPC C12N 15/101

(57) ABSTRACT

A synchronous coefficient of drag alteration method and system for concentrating and purifying nucleic acids from a sample containing unwanted molecules and contaminants are provided. The method includes providing a layer of artificial gel or sieve filled with liquid and into which molecules including nucleic acids are introduced from a sample liquid by applying a steady electric field across a boundary between the artificial gel layer and the sample liquid containing the molecules including nucleic acids; providing a plurality of electrodes spaced apart around circumference of the artificial gel layer and for delivering a pre-programmed sequence of voltage waveforms to establish a primary electric field driving molecules within the artificial gel layer and a secondary electric field altering electrophoretic mobility of molecules within the artificial gel layer. The nucleic acids alone are spatially separated from the unwanted molecules and contaminants to be relatively purified and enriched.

### CROSS-REFERENCE TO RELATED APPLICATIONS

The subject application claims the benefit of International PCT Application No. PCT/IB2024/000192, filed on Apr. 18, 2024, which claims the benefit of U.S. Provisional Application Ser. No. 63/496,956, filed Apr. 19, 2023, which is incorporated herein by reference in its entirety.

### BACKGROUND

Artificial gels are used for nucleic acid purification from contaminants through selective electrophoretic focusing. Herein, the artificial gels refer to porous structures that, unlike conventional polymer gels, can withstand high electric field and high temperature so that electrophoresis of molecules can occur under high voltages for fast separation and purification of nucleic acids from contaminants. Artificial gels contain microscopic pores that are in the range of micrometers to nanometers and may or may not be uniform in size. Artificial gels can be fabricated through various methods including the assembly of colloids, droplets or bubbles (foam) or the synthesis of nanowires/nanotubes or cleanroom micro/nanofabrication techniques that may involve lithography. Molecules including nucleic acids are injected into an artificial gel filled with a running buffer under an electric field externally applied. Nucleic acids inside the artificial gel are selectively focused (concentrated) into a specific zone being spatially separated from most other unwanted molecules and contaminants under an alternating field that drives molecules and a further alternating field that alters the mobility of the molecules. Nucleic acids upon concentration and purification can be collected from the artificial gel for downstream use. They can be also directly detected inside the artificial gel either before or after subjecting them to various processes including polymerase chain reaction (PCR) and loop-mediated isothermal amplification (LAMP). The PCR or LAMP process may also be performed inside one of the fluidic reservoirs supplying the artificial gel layer. In other words, these processes are not necessarily performed directly inside the artificial gel layer itself, but rather within a fluidic reservoir associated with the artificial gel layer within the same chip.

Sample preparation has remained a major “grand challenge” after decades of research and has continued to present a bottleneck in the complete automation of nucleic acid analysis. Automation through the miniaturization of sample preparation into a fluidic microchip has seen a number of advancements. Miniaturization of nucleic acid purification through microfluidics dates back to 1998, when Christel et al. reported a system for point-of-use rapid nucleic acid (NA) analysis [1]. The authors described a microfluidic chip featuring an array of silicon micropillars to purify DNA from clean samples based on solid phase extraction (SPE), a commonly used method in many commercial NA purification kits. These kits utilize some sort of silica-gel, glass matrix, membrane column or beads and work based on the principle that NA reversibly binds to glass or silica-like surface in the presence of chaotropic salts such as GuHCl. Bound NA desorbs from the surface in a low-salt wash applied after removing the impurities in an alcohol-based wash. Similarly, silicon pillars once oxidized offer a silica-like surface which can capture DNA presented in GuHCl. Cady et al. enhanced the binding capacity of the oxidized silicon micropillars from 40 to 82 ng/cm² and purified DNA from intact cells [2]. The same approach has been used by others in a fully integrated microchip that can extract and purify genomic DNA directly from whole blood [3,4]. To further increase binding capacity, Landers and colleagues showed microchannels with packed columns of silica beads or particles, porous silica sol-gel columns, and silica bead/sol-gel hybrid columns [5-7]. The researchers have explored additional solid phases to avoid chaotropic salts and organic solvents which are known to inhibit PCR, the downstream NA amplification step [8-10].

While the aforementioned studies pioneered the on-chip NA purification, they adopted SPE in a miniaturized format, thereby inheriting some of its drawbacks. SPE protocol albeit its excellent efficiency involves multiple wash steps with chemistry that may interfere with downstream analyses. As SPE relies on selective affinity between NA and a solid phase, it performs poorly in the presence of contaminant molecules that show similar affinity to solid phase [11]. Such contaminants carry through and have to be removed in a precipitation step. Moreover, these contaminants, when abundant, could foul the surface and prevent the binding of NA, greatly reducing the yield. Already, the yield of SPE does not scale well with reduction in surface area, which puts miniaturized platforms at a disadvantage compared to commercial kits. The colossal surface area of spin columns and magnetic beads in those kits are unmatched by the surface area boost brought by microchannels featuring packed columns or micropillars. Also, the bind-wash-elute workflow requires fluidic valving for rapid switching with minimal dead volume to prevent chemical carry over between these steps, which increases not only the design and fabrication complexity but also the complications during operation. As such, simplified microchip concepts for sample preparation are needed particularly for the preparation of long DNA, given the future potential of long-read sequencing technologies in healthcare.

In recent years, this area of research has been revived with innovative use of known and established techniques such as electrophoresis [12-15], isotachophoresis (ITP) [16-21] and those that found renewed interest such as ion concentration polarization (ICP) [22-25] and entropic trapping [26]. In those demonstrations, abandoning the surface-binding approach (SPE) has proven to be effective, and already resulted in commercial products such as Ionic Purification System by Purigen Biosystems.

Recently emerged as a NA purification technique, ITP is an analytic technique for selective separation and concentration of ionic analytes. It is characterized by a discontinuous buffer system with the analytes of interest separated into tightly focused bands between a zone of fast leading electrolyte (LE) and a zone of slow trailing electrolyte (TE) under a steady field. Leveraging the high anionic mobility of NAs, ITP has been applied for the isolation of DNA from human blood, serum, and plasma [16-18], and the purification of RNA from bacteria in urine [19], and blood [20], as well as small RNA from total RNA in kidney cells [21]. However, preparative ITP is highly sensitive to the temperature (joule heating) and chemistry of the TE/LE, and as such requires fine tuning of the ion composition, including ion mobility, concentration, volume, as well as pH for different samples. Furthermore, potential pH shift due to electrochemical reactions poses challenges to high-throughput processing.

ICP has received broad attention in microfluidics initially for rapid enrichment of biomolecules in particular proteins and also water desalination [23]. The method leverages the selective removal of cations through a cation-exchange membrane or nanochannel(s) under a steady electric field and the subsequent ion-depletion zone induced in the microchannel with a greatly enhanced electric field. This field acts as an electric-force barrier and stacks anionic species delivered by an electroosmotic flow in a continuous-flow preconcentration at a site where electrophoresis and electroosmosis may balance each other. An initial attempt of applying the technique for purifying NA from a complex sample (serum) has faced several major challenges [24]. These have been recently addressed by introducing a pressure-driven flow to the microchannel, which allowed proteins to stack in the vicinity of the peak of the electric-force barrier where they can easily leak downstream by diffusion [25]. Through modulating the applied pressure, NAs of high electrophoretic mobility become selectively trapped, while the majority of molecules of low electrophoretic mobility are removed. Although the enrichment increases with the applied pressure, further increase beyond a threshold pressure leads to the loss of NAs along with proteins and contaminants. Moreover, the threshold pressure is sensitive to protein concentration, ionic strength, and viscosity, and thus requires a sample-by-sample calibration to decide the optimal experimental conditions.

Entropic trapping for NA purification has only been recently reported, albeit as a downstream process for purifying long DNA from short DNA in a clean buffer [26]. Short DNA overcomes entropic barriers under an electric field strength, which is insufficient to drive long DNA over those barriers. This leaves long DNA to remain trapped while short DNA is being removed. In microfluidics, entropic barriers are formed at the entrance of a confined space, typically a nanoslit (1D confinement), and long DNA must undergo conformational changes to enter in. A series of well and slit regions has been initially applied for the size-based sorting of macromolecules, including DNA, based on entropic trapping [27]. Also referred to as the slit-well (SW) array, this design features hundreds of entropic barriers with each retaining DNA for a finite duration under a moderate field strength. Increasing the field strength increases the separation speed and quality, but only to the extent that the trapping starts to fail, with all molecules migrating at the same speed, regardless of size. Such a problem is inherent to slits which make relatively weak entropic barriers. Even in the reported slit-based purification, increasing the electric field strength to 12 V/cm enhances the filtration selectivity

with 90% of short DNA being removed, but at the expense of the long DNA yield [26]. Thus, SW array may not be ideal for rapid extraction of DNA from a complex sample at an acceptable purity.

Electrophoretic focusing and purification of NA exploits a novel multidimensional transport mechanism in a method called synchronous coefficient of drag alteration (SCODA) [12]. It can concentrate DNA molecules in an agarose gel at a region free of electrodes to avoid any electrochemical damage. This is achieved with the simultaneous application of a uniform dipole electric field rotating at a particular angular frequency and a quadrupolar electric field oscillating at twice that angular frequency. Either field imparts no net drift to molecules except those whose drag (mobility) is field dependent. Thus, DNA molecules migrate on average away from the nearest electrode toward a central region while other charged molecules such as proteins simply move in circles under zero time-averaged fields. The SCODA has been proven effective to isolate DNA from humic acids, a panel of contaminants abundant in soil [13], extract DNA from challenging oil sands [13], and separate DNA from PCR inhibitors for forensic applications [14]. It can yield long (high molecular weight) DNA with high purity suitable for library construction and metagenomics analysis. The method in its current format, however, requires several hours in total run time [15], which can be considerably shortened by adopting artificial sieves (the invention) that can sustain fairly large fields. There continues to be a need in the art for improved designs and techniques for methods and systems for nucleic acid purification.

### SUMMARY

According to an embodiment of the subject invention, a synchronous coefficient of drag alteration (SCODA) method for concentrating and purifying nucleic acids from a sample containing unwanted molecules and contaminants is provided. The method comprises providing a layer of artificial gel or sieve filled with liquid and into which molecules including nucleic acids are introduced from a sample liquid by applying a steady electric field across a boundary between the artificial gel layer and the sample liquid containing the molecules including nucleic acids; and providing a plurality of electrodes spaced apart around circumference of the artificial gel layer and connected to at least one external power supply for delivering a pre-programmed sequence of voltage waveforms to establish a primary electric field driving molecules within the artificial gel layer and a secondary electric field altering electrophoretic mobility of molecules within the artificial gel layer; wherein the plurality of electrodes is configured to generate a pre-programmed sequence of voltage waveforms such that the nucleic acids alone are driven to a certain zone in the artificial gel layer where they are converged and spatially separated from the unwanted molecules and the contaminants to be relatively purified and enriched. The primary electric field and secondary electric field are established in the artificial gel layer concurrently. The sample introduction and nucleic acid enrichment and purification are performed concurrently. Moreover, the artificial sieve layer is made of silicon-based, silica-based, or polymer-based insulating or dielectric materials through cleanroom processes involving lithography. Alternatively, the artificial sieve layer is made of assembled layer(s) of colloids, bubbles or foam, droplets, nanowires, or nanotubes. The artificial sieve layer is configured to sustain high voltage and/or high temperature. The SCODA method can further comprise collecting the nucleic acids enriched and purified from the unwanted molecules and the contaminants from the artificial sieve layer for downstream use. The SCODA method can be followed by applying PCR or LAMP to the nucleic acids enriched and purified from the unwanted molecules and contaminants without collecting the nucleic acids from the artificial gel layer. Furthermore, PCR or LAMP can be performed inside one of fluidic reservoirs after transporting enriched and purified nucleic acids into the fluidic reservoir. The amplified target sequence or sequences are detected in real time or following the amplification process without collecting the amplified products from the artificial gel layer. A reaction temperature is controlled by external means and hardware interfacing with the artificial gel layer. The detection of the amplified products is achieved by electrical or optical hardware interfacing with the artificial gel layer or a fluidic reservoir associated with the artificial gel layer.

According to another embodiment of the subject invention, a synchronous coefficient of drag alteration (SCODA) system for concentrating and purifying nucleic acids from a sample containing unwanted molecules and contaminants is provided. The SCODA system comprises a layer of artificial gel or sieve filled with liquid and into which molecules including nucleic acids are introduced from a sample liquid by applying a steady electric field across a boundary between the artificial gel layer and the sample liquid containing the molecules including nucleic acids; a plurality of electrodes spaced apart around circumference of the artificial gel layer and connected to at least one external power supply for delivering a pre-programmed sequence of voltage waveforms to establish a primary electric field driving molecules within the artificial gel layer and a secondary electric field altering electrophoretic mobility of molecules within the artificial gel layer; wherein the plurality of electrodes are configured to generate a pre-programmed sequence of voltage waveforms such that the nucleic acids alone are driven to a certain zone in the artificial gel layer where they are converged and spatially separated from the unwanted molecules and the contaminants to be relatively purified and enriched. The primary electric field and secondary electric field are established in the artificial gel layer concurrently. The sample introduction and nucleic acid enrichment and purification are performed concurrently. The artificial sieve layer is made of silicon-based, silica-based, or polymer-based insulating or dielectric materials through cleanroom processes involving lithography. Alternatively, the artificial sieve layer is made of assembled layer(s) of colloids, bubbles or foam, droplets, nanowires, or nanotubes. Further, the artificial sieve layer is configured to sustain high voltage and/or high temperature. The nucleic acids enriched and purified from the unwanted molecules and the contaminants are collected from the artificial sieve layer for downstream use. Alternatively, PCR or LAMP can be performed on the enriched and purified nucleic acids from the unwanted molecules and contaminants, without collecting the nucleic acids from the artificial gel layer. The amplified target sequence or sequences are detected in real time or following the amplification process without collecting the amplified products from the artificial gel layer.

In one embodiment, the amplification process may take place in one of the fluidic reservoirs associated with the artificial gel layer following the electrokinetic transport of the purified nucleic acids into the fluidic reservoir.

### BRIEF DESCRIPTION OF THE DRA WINGS

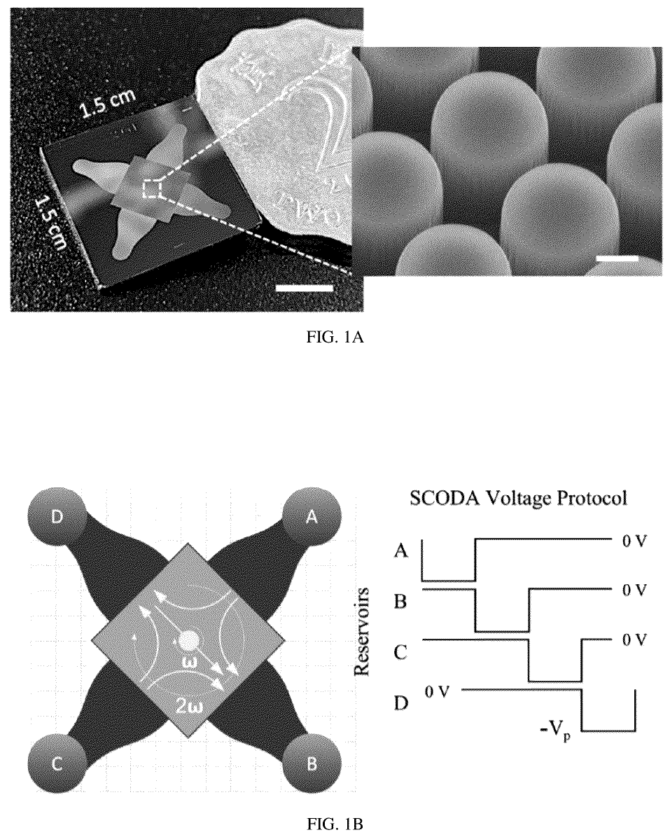

FIG. 1A is an image of microfluidic chip featuring the sieve (shown without cover; scale bar: 0.5 cm). Inset: scanning electron microscopy image of the sieve (scale bar: 2 μm).

FIG. 1B is a schematic representation of the operation in which a single period of the voltage protocol applied to fluidic reservoirs through wire electrodes to induce dipole and quadrupole electric fields rotating at angular frequencies of ω and 2ω, respectively, each electrode being periodically pulsed for a step duration that is equivalent to a quarter of one period, according to an embodiment of the subject invention.

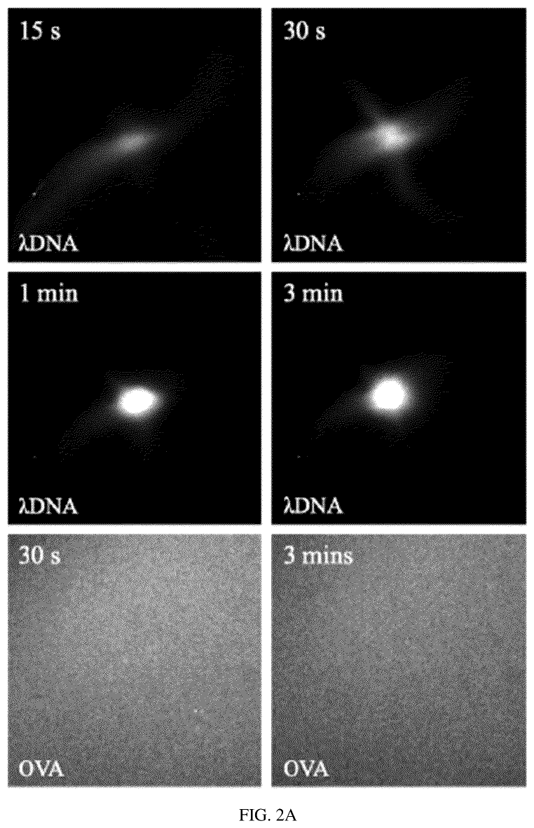

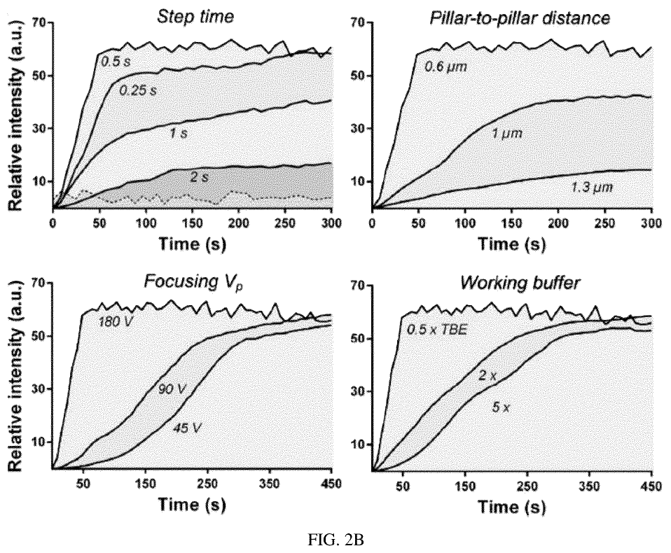

FIG. 2A shows time-lapsed fluorescence images demonstrating the sieve center during SCODA focusing of YOYO-stained λDNA (9 ng/μL) in the presence of Alexa-labeled OVA (2 mg/mL) in 0.5×TBE buffer. Activation (unless otherwise stated): 180 Vₚ applied for a step duration of 0.5 s across a sieve with a pillar-to-pillar spacing of 0.6 μm and filled with 0.5×TBE buffer, according to an embodiment of the subject invention.

FIG. 2B shows time traces of fluorescence intensity levels in the sieve center registered by λDNA under stated step duration (dashed line indicates time trace of OVA's fluorescence intensity with 0.5 s step duration), peak voltage Vₚ, pillar-to-pillar spacing, and working buffer. Activation (unless otherwise stated): 180 Vₚ applied for a step duration of 0.5 s across a sieve with a pillar-to-pillar spacing of 0.6 μm and filled with 0.5×TBE buffer, according to an embodiment of the subject invention.

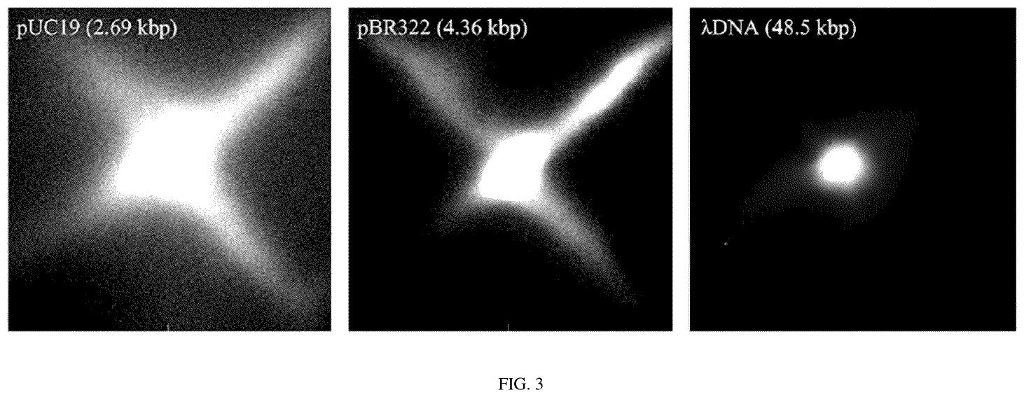

FIG. 3 shows dependence of SCODA focusing on the DNA length shown through YOYO-stained plasmid vectors pUC19, pBR322, and λDNA obtained after 3 min of SCODA focusing (Activation: 180 Vₚ for a step duration of 0.5 s. DNA concentration: 9 ng/μL in 0.5×TBE), according to an embodiment of the subject invention.

FIG. 4 shows fluorescence images demonstrating gradual removal of Alexa-labeled OVA off the sieve (upper row) during electrophoretic wash steps introduced between SCODA focusing cycles and subsequent focusing and defocusing of YOYO-stained λDNA (lower row), wherein dashed circles mark the initial position of focused λDNA molecules, according to an embodiment of the subject invention.

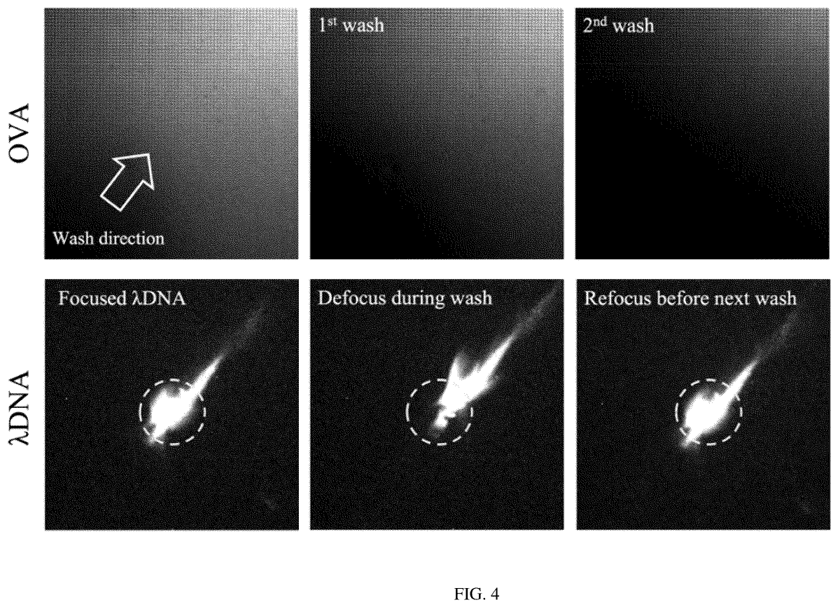

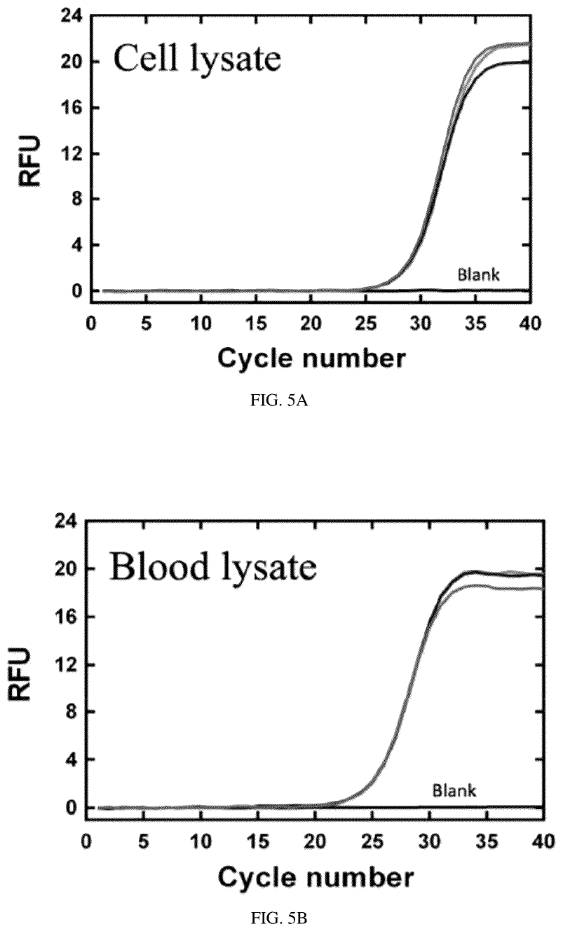

FIG. 5A shows results of qPCR analysis demonstrating the amplification curves from independent experiments on fractions collected after being SCODA concentrated and purified from cell lysate (10× dilution), according to an embodiment of the subject invention.

FIG. 5B shows results of qPCR analysis demonstrating the amplification curves from independent experiments on fractions collected after being SCODA concentrated and purified from whole blood lysate (20× dilution), according to an embodiment of the subject invention.

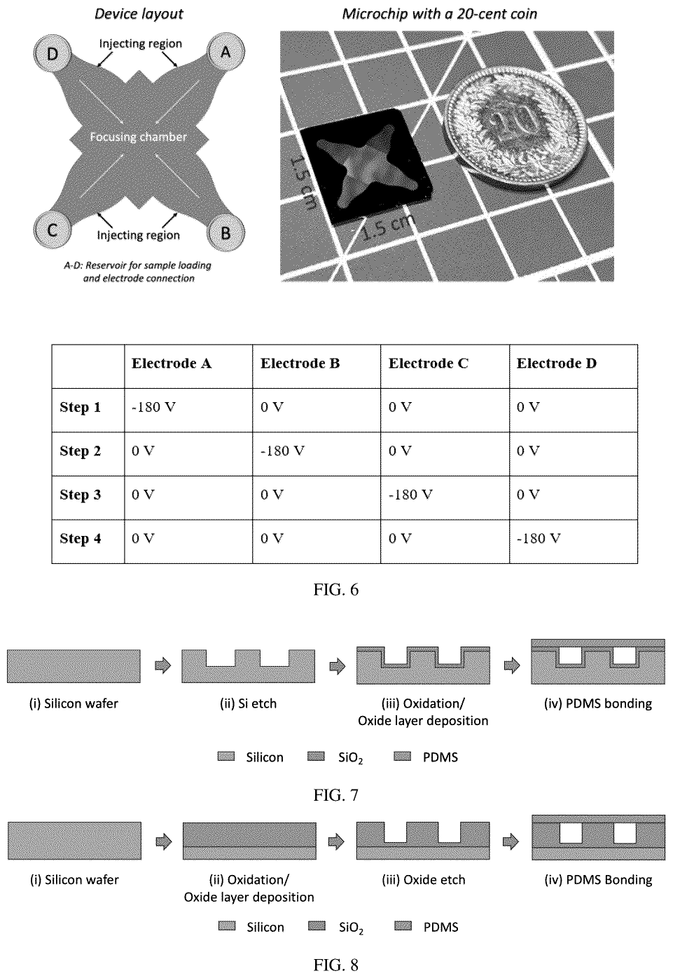

FIG. 6 shows (upper left) schematic representation of the design layout of the DNA purification microchip featuring a focusing chamber consists of a micro-pillar array (artificial gel) surrounded by four injecting regions; and (upper right) a photograph of the microchip (1.5 cm by 1.5 cm) placed next to a 20-cent coin, according to an embodiment of the subject invention. Table below shows an example of a sequence of voltages applied to the four wire electrodes placed in the reservoirs A, B, C, and D in four consecutive steps. The voltage levels in each step are maintained for a specific duration (for example, 250 ms) before moving to the successive step.

FIG. 7 describes one method of fabrication process of artificial gels featuring the micro-pillar array using the cross-sectional diagrams of a silicon wafer: (i) starting Si wafer, (ii) Si etch after lithographic patterning, (iii) thermal oxidation and/or oxide layer deposition, and (iv) bonding of a polydimethylsiloxane (PDMS) slab cover, according to an embodiment of the subject invention.

FIG. 8 describes another method of fabrication process of artificial gels featuring the micropillar array using the cross-sectional diagrams of a silicon wafer: (i) starting Si wafer, (ii) oxidation and/or oxide layer deposition, (iii) oxide etch after lithographic patterning and (iv) bonding of a PDMS slab cover, according to an embodiment of the subject invention.

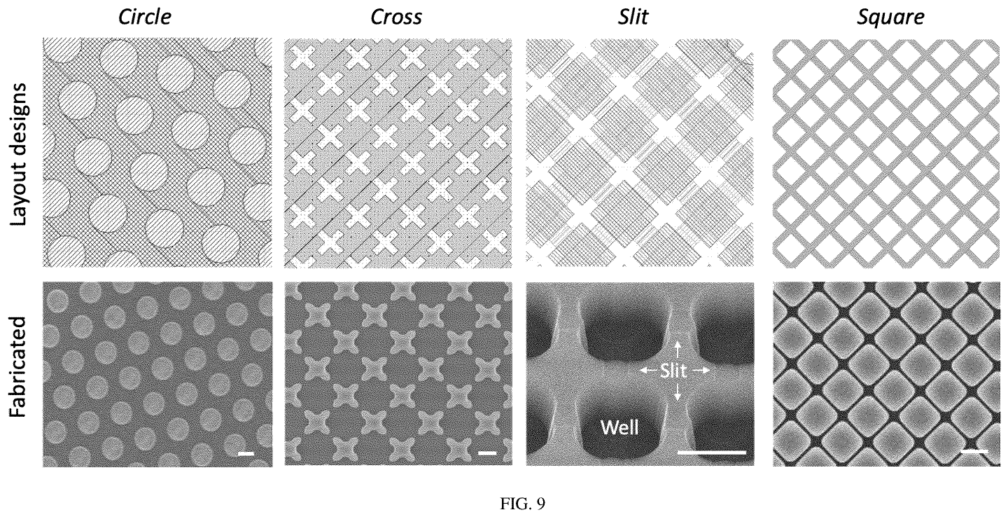

FIG. 9 shows design layouts and corresponding SEM images of the fabricated artificial gels with micropillar arrays consisting of micropillars of various shapes including circle, cross, cross with a slit, and square, according to an embodiment of the subject invention. (Scale bars: 5 μm)

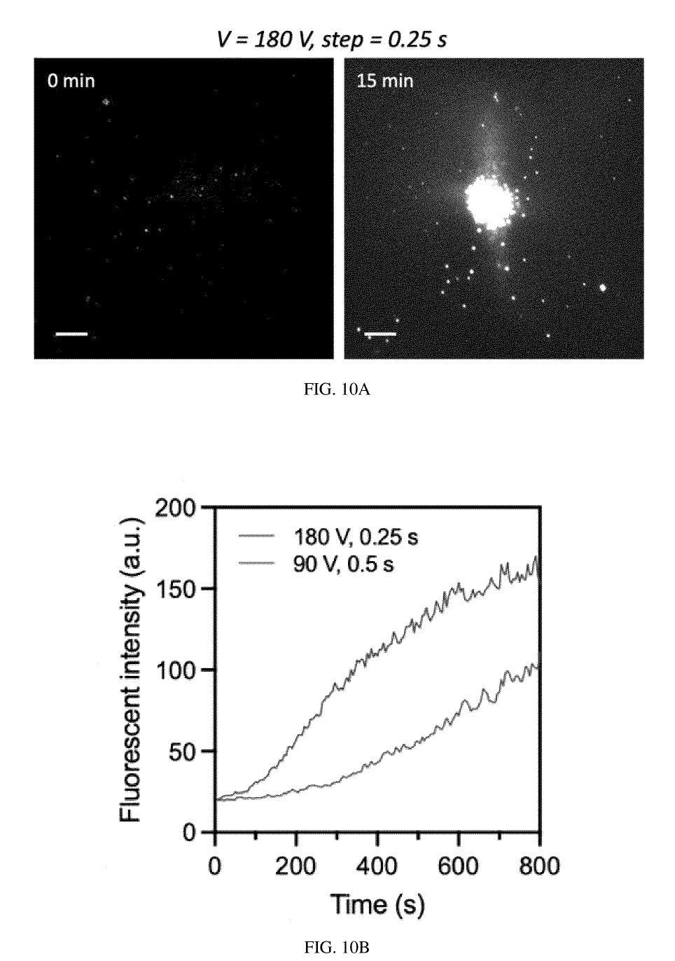

FIG. 10A depicts the fluorescent images from the center of an artificial gel with cross-shaped pillars set 2 μm apart and filled with a buffer containing fluorescent-tagged lambda DNA before and after the application of a pre-programmed rotating electric field for 15 mins (180 V applied in rotation with a step interval of 250 ms) (Scale bars: 200 μm).

FIG. 10B shows the time evolution plot of the fluorescent intensity at the gel center under two distinct voltage protocols: 180 V applied in rotation with a step interval of 250 ms; and 90 V applied in rotation with a step interval of 500 ms.

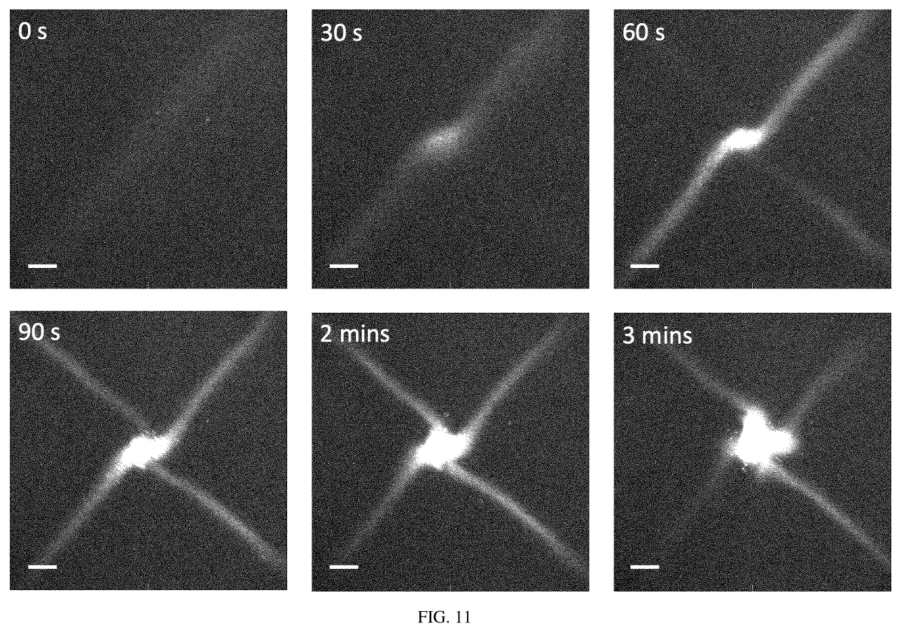

FIG. 11 shows the focusing of fluorescent-stained lambda DNA within 3 minutes with the fluorescent images depicting the center of an artificial gel (with round pillars set 600 nm apart) at different time points since the onset of the voltage protocol (180 V applied in rotation with a step interval of 500 ms) (Scale bars: 200 μm).

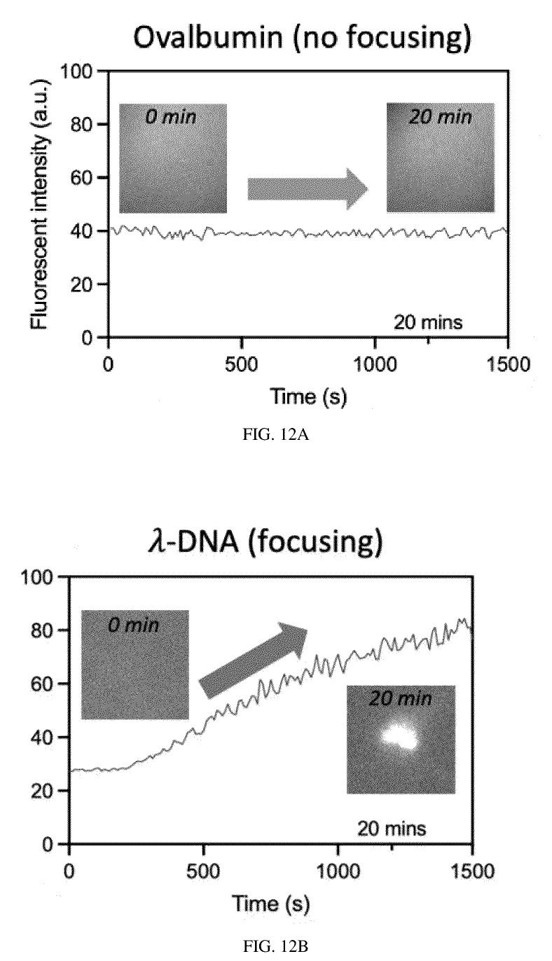

FIG. 12A shows that no focusing can be observed for ovalbumin (used as a model contaminant protein) during focusing and purification of nucleic acids from other molecules or contaminants. The inset images correspond to the microchip center before and after applying 20 mins of rotating electric field, according to an embodiment of the subject invention.

FIG. 12B shows focusing of lambda DNA to the microchip center in the same solution. The inset images correspond to the microchip center before and after applying 20 mins of rotating electric field, according to an embodiment of the subject invention.

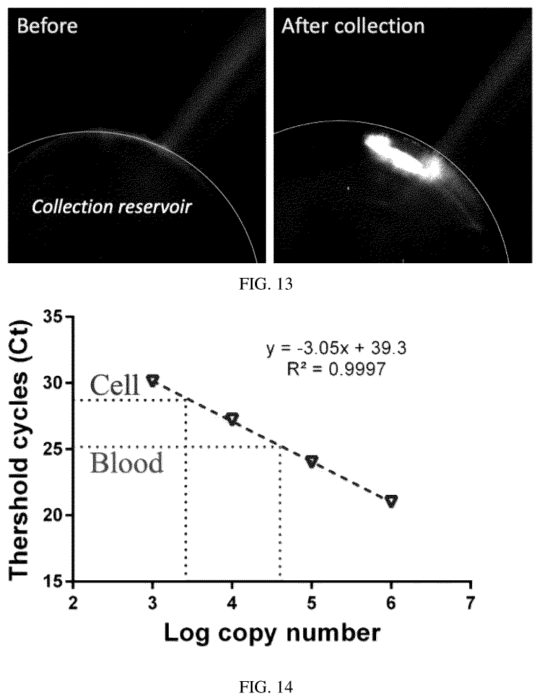

FIG. 13 shows electrophoretic transport and collection of YOYO-labelled λDNA in reservoir C after 300 cycles of SCODA focusing and 15 intermediate wash steps, wherein Activation: 180 Vₚ for a step duration of 0.5 s and Buffer: 0.5×TBE, according to an embodiment of the subject invention.

FIG. 14 is a cycle threshold (CT) plot for the PCR amplification based on standards, wherein the standard curves show a high correlation (R²>0.999) between the log copy number of the target gene and the CT value obtained from the PCR reaction, according to an embodiment of the subject invention.

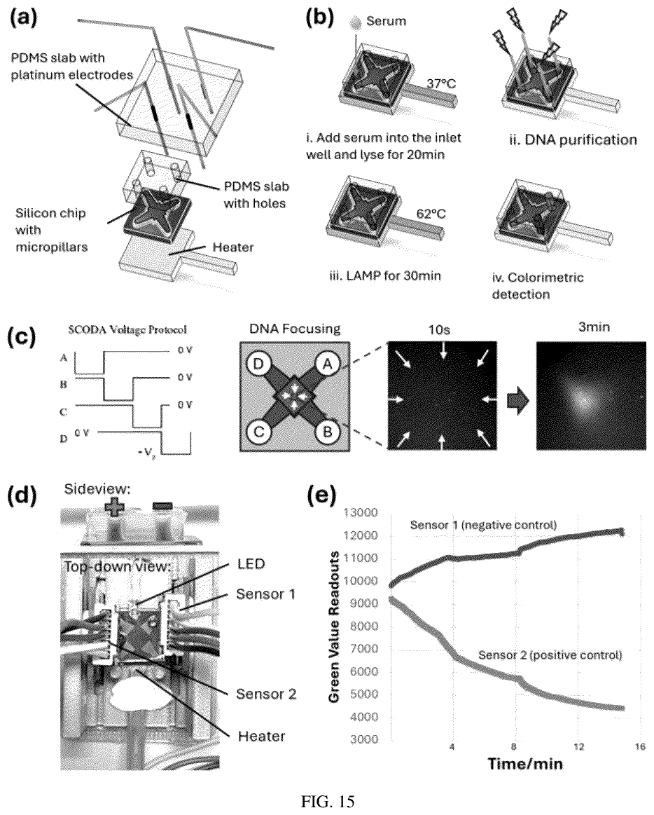

FIG. 15 shows (A) an exploded, schematic view of the microfluidic chip used in an integrated MRSA detection system. (B) shows steps of a workflow of the integrated system for MRSA detection. (C) shows a schematic of a voltage protocol for nucleic acid purification process by synchronous coefficient of drag alteration (SCODA) on the chip. (D) shows a microfluidic chip, with the top panel showing a post-LAMP side view showing reservoirs containing a positive sample and a no-template control (negative sample); the lower panel shows a top-down view of the chip setup for real-time monitoring of colorimetric LAMP reactions. (E) shows a line graph plot illustrating the real-time colorimetric transition during the 15-min LAMP reactions.

### DETAILED DESCRIPTION

Embodiments of the subject invention are directed to a synchronous coefficient of drag alteration (SCODA) method and systems for concentrating and purifying nucleic acids from a sample containing unwanted molecules and contaminants.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention pertains. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

When the term “about” is used herein, in conjunction with a numerical value, it is understood that the value can be in a range of 90% of the value to 110% of the value, i.e. the value can be +/−10% of the stated value. For example, “about 1 kg” means from 0.90 kg to 1.1 kg.

In describing the invention, it will be understood that a number of techniques and steps are disclosed. Each of these has individual benefits and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

### EXAMPLE 1

SCODA can yield long (high molecular weight) DNA with high purity for library construction and metagenomics analysis. The method in its current format, however, requires several hours in total run time. According to embodiments of the subject invention, it is demonstrated that this duration can be greatly reduced by adopting an artificial sieve sustaining large fields as shown in FIG. 1A.

Synchronous coefficient of drag alteration refers to a multidimensional transport mechanism where a net drift of molecules is achieved under a zero-time-average alternating motive force by perturbing their drag coefficient synchronously with the applied force. An electrophoretic form of the method is often applied to focus and purify nucleic acids in a gel under rotating electric fields. However, this method requires lengthy operation due to the use of limited field strengths. Here, using DNA as target molecules, we demonstrate that the operation time can be reduced from hours to minutes by replacing polymer gel with a microfabricated artificial sieve. We also describe an electrophoretic protocol that facilitates the collection of purified DNA from the sieve, which is shown to yield amplifiable DNA from crude samples including the lysates of cultured cells and whole blood. The sieve can be further equipped with nucleic acid amplification and detection functions for a point-of-care diagnostic application.

### Materials and Methods

### Materials

λDNA and Tris/Borate/EDTA (TBE) buffer were obtained from Nippon Gene (Toyama, Japan). Plasmid vectors pUC19 and pBR322 DNA and ovalbumin (OVA) Alexa Fluor 488 Conjugate were purchased from Thermo Fisher Scientific (Waltham, MA). DNA intercalating dye YOYO-1, YOYO-3, and human serum (from human male AB plasma, USA origin, sterile-filtered) were procured from Sigma-Aldrich (Burlington, MA). Primers for quantitative PCR (qPCR) were synthesized by Integrated DNA Technologies. LightCycler 480 SYBR Green I Master was obtained from Roche Diagnostics GmbH (Mannheim, Germany). Lysis buffer and protease K were purchased from Qiagen (Hilden, Germany). PDMS was acquired from Dow Corning (Midland, MI). Performance Optimized Polymer-6 (POP-6) was obtained from Applied Biosystems (Foster City, CA). All solutions were prepared using nuclease-free water from Thermo Fisher Scientific (Waltham, MA).

### Microfabrication

The micropillar arrays were patterned on 4-in. silicon wafers through a single-mask photolithography process, followed by deep reactive ion etching to a depth of 20 μm with a pillar-to-pillar spacing of 1.5 μm. The pillar-to-pillar spacing was reduced through thermal oxidation. The arrays and fluidic channels were enclosed from above with polydimethylsiloxane (PDMS) slabs featuring fluidic inlet and outlet ports and electrical access via bonding to the silicon substrate after surface activation in oxygen plasma.

### Sample Preparation

The DNA samples were stained with the intercalating dyes YOYO-1 or YOYO-3 at a dye-to-base pair ratio of 1:10 in TBE buffer. The stained DNA samples as well as fluorescently-tagged OVA were further diluted in 1×TBE buffer to the desired concentrations. To create a mock sample, YOYO stained λDNA was mixed with fluorescently-labeled OVA protein as a contaminant. The cell lysate was prepared from human embryonic kidney cells (HEK 293T) cultured at 37° C. in a humidified incubator aerated with 5% CO2 in Dulbecco's modified Eagle medium (DMEM), supplemented with 10% fetal bovine serum and 1% penicillin/streptomycin. Blood lysate was prepared from whole blood provided by the campus animal facility.

### Experimental Setup

The chip was filled with running buffer (Tris/Borate/EDTA buffer containing 0.5% v/v POP-6 to suppress electroosmosis). The voltages sourced from a high voltage sequencer (HVS448-8000D, LabSmith Inc. Livermore, CA) were applied via four platinum wire electrodes placed in reservoirs surrounding the artificial sieve. A 10-minute pre-run was conducted by sequentially applying a peak voltage (Vₚ) of 500 Vₚ to each electrode for 100 seconds to remove bubbles trapped in the sieve until the current through the device became stable. The sieve was then filled with the input sample by an electrophoretic injection. For DNA focusing, in one example, the electrodes were sequentially activated at an optimal peak voltage of 180 Vₚ for a step duration of 0.5 second. To purify the DNA from contaminants, an electrophoretic wash step lasting 1 second was applied following DNA focusing to remove contaminants from the sieve. The purified DNA was moved to the collection reservoir by electrophoresis.

qPCR Analysis

PCR amplification was performed in triplicate on a LightCycler 480 Instrument II (Roche). The reaction mixture comprises 5 μL of DNA and 20 μL of master mix (LightCycler 480 SYBR green I master, PCR grade nuclease-free water, forward and reverse primers). The PCR running protocol was set at 95° C. for 10 minutes for λDNA, followed by 40 cycles of denaturation at 95° C. for 10 seconds, annealing at 54° C. for 10 seconds, and extension at 72° C. for 10 second. For the glyceraldehyde-3-phosphate dehydrogenase (GAPDH) gene, it was set at 50° C. for 2 minutes and then 95° C. for 10 minutes, followed by 40 cycles of denaturation at 95° C. for 15 seconds, annealing and extension at 60° C. for 60 seconds. The absolute concentration of the target gene was calculated by generating a standard curve from the GAPDH qPCR template standard (Origene Technologies, Rockville, MD).

### DNA Quantification

The quantification of doublestranded DNA (dsDNA) in both input and output samples was performed using a NanoDrop 3300 Fluorospectrometer (Thermo Scientific) in conjunction with the PicoGreen assay (Invitrogen Molecular Probes, ThermoFisher Scientific, Waltham, MA), as per the manufacturer's protocol. Excitation was performed at 480 nm, and fluorescence emission intensity was measured at 525 nm.

### Protein Analysis

The protein concentration of samples was measured using the Pierce BCA Protein Assay Kits (Thermo-Fisher Scientific). The samples were mixed with the BCA reagent and incubated for 30 min at 37° C. The absorbance was measured at 562 nm using a Molecular Devices FlexStation 3 Multimode Microplate Reader (Molecular Devices, Sunnyvale, CA). The protein concentrations were calculated based on a calibration curve generated using bovine serum albumin standards. Each sample and standard point was measured in triplicate.

### Results

FIG. 1A shows a picture of a silicon chip featuring the sieve. The sieve is an array of 5 μm-diameter, 20 μm-tall thermally oxidized silicon pillars with a pillar-to-pillar spacing of 0.6 μm (FIG. 1A, inset). FIG. 1B describes a voltage protocol for inducing rotating dipole and quadrupole fields that preferentially focus DNA into the sieve center and thus result in purification of DNA from contaminants. The principle of SCODA is detailed in previous publications [12, 13] and is summarized here briefly. While electrophoretic mobility is nearly constant for most molecules, it shows linear field dependence for DNA as predicted by the biased reptation with fluctuations model and experimentally verified by agarose gel electrophoresis [28, 29]. The field dependence is more pronounced for longer DNA fragments than shorter ones and leads to a quadratic dependence of DNA velocity on field. Thus, applying a dipole field rotating at ω results in a frequency-doubled velocity component oscillating at 2ω. When a mobility-modulating quadrupole field rotating at 2ω is concurrently applied, it gives rise to an average drift velocity of DNA pointing to the center of the field pattern. This radial drift velocity drives the preferential focusing of DNA and is proportional to the dipole and quadrupole field strengths as well as the radial distance between the DNA and the center of the field pattern.

FIGS. 2A and 2B show SCODA focusing of YOYO-stained λDNA in a mock sample containing fluorescently-labeled OVA protein as the contaminant and initial uniform dispersion across the sieve through electrophoretic injection. Electrophoretic injection of the sample into the sieve introduces only negatively charged contaminants along with DNA. Fluorescence images reveal that λDNA can be focused into the sieve center within a minute with each of the four electrodes sequentially activated at 180 Vₚ for a step duration of 0.5 seconds (FIG. 2A). Meanwhile, no observable change in the OVA distribution profile can be seen.

In FIG. 2B, the influence of various conditions on the focusing dynamics of λDNA is shown through the time traces of fluorescence intensity levels averaged within the focused spots at the center of the sieve.

A steep focusing trace is noted for an activation at 180 Vₚ (˜330 V/cm average field strength) applied for a step duration of 0.5 s in combination with 0.5×TBE working buffer across the sieve featuring 0.6 μm pillar-to-pillar spacing. The trace saturates, and there is no further increase in the focused amount beyond a 1 min focusing as the amount is limited by the sieve capacity. The concentration of DNA in the focused spot is estimated to be about 1 μg/μL, yielding an enrichment factor of about 100. Reaching saturation takes a longer time for SCODA focusing with a reduced peak voltage or an increased step duration. The former is understandable due to the reduced field strengths, whereas the latter is likely due to the lengthened trajectory of λDNA. The DNA oscillates while drifting toward the center. The oscillations become larger with the increased step duration. However, reducing step duration below 0.5 s slows the focusing because switching the activation faster than the reorientation time of reptating λDNA molecules likely inhibits their motion. Increased ionic strength of the working buffer and increased pillar-to-pillar spacing of the sieve also lead to reduced field strengths which can retard the focusing process.

FIG. 3 shows the size dependence of DNA focusing. Similar to λDNA, plasmid vectors pUC19 DNA (2.69 kbp) and pBR322 DNA (4.36 kbp) can be focused on the sieve center under 180 Vₚ applied for a step duration of 0.5 second. A major difference between the results is that the larger the DNA molecules, the smaller is the focused spot size because diffusion works against the focusing and small molecules diffuse readily. We have managed to focus dsDNA as short as 500 bp in the sieve (not shown). In contrast to the previously reported gel-based device, which required 2 hours to focus λDNA from an initial spot size of 1 cm under an electric field strength of ˜52 V/cm applied with a period of 12 seconds [12], our artificial sieve-based device can achieve SCODA focusing in less than 1 min (with an initial λDNA suspension in 0.55 cm×0.55 cm area) by applying a stronger electric field of about 330 V/cm with a period of 2 second. Different from polymer gels, our silicon-based artificial sieve can sustain strong electric fields while limiting the current and thus avoiding joule heating. Our artificial sieve device also demonstrated its versatility for DNA with a wide length range from 500 bp to lambda DNA and genomic DNA, unlike the gel-based device, which required different agarose concentrations (>1% for short DNAs and 0.4% for high-molecular-weight DNA) for different DNA lengths [13]. The focused DNA must be harvested from the sieve center in purified form for subsequent analysis. Placing a reservoir in the sieve center alters the electric field distribution and thus disrupts focusing. Alternatively, DNA can be harvested from one of the existing reservoirs via electrophoretic transport. Before collection, however, the sieve must be cleared of contaminants. We introduced electrophoretic wash steps into the focusing protocol to gradually wash contaminants out of the sieve. For the wash, we applied a pulse of 180 Vₚ for 1 second to reservoir C (FIG. 1B) after a SCODA focusing cycle, which comprises a plurality of rounds, for example, 20 rounds of activation of the 4 electrodes in sequence.

FIG. 4 shows fluorescence images of the sieve from this process where the first and second wash steps gradually removed OVA. Although λDNA molecules get slightly defocused during the wash steps, they become refocused during the subsequent focusing cycles.

λDNA can be transported under a steady bias to the collection reservoir after being concentrated and purified in the sieve center through SCODA focusing (FIG. 13). This enables the collection of purified DNA for subsequent analysis. The consistent size and location of the focused spot from run to run enabled the collection of purified DNA for subsequent analysis, without the need to stain molecules. The following tests were performed blindly, relying on the reliability of the focusing process. DNA yield, as measured using the PicoGreen assay, is about 72%±18%, based on a collection amount of 1.50±0.15 ng of λDNA in output reservoir after SCODA purification (300 cycles of focusing and 15 intermediate wash steps), starting from an input amount of 2.10±0.40 ng. Protein rejection tests using human serum samples spiked with λDNA indicate a rejection ratio of at least 2200 with protein amount reduced from 55±0.5 mg/mL in human serum (input sample) to below 25 μg/mL (the detection limit) in the purified DNA sample (after 300 cycles of SCODA focusing and 15 intermediate wash steps) according to the Pierce protein assay. SCODA purification results of various sample types from the sieve are listed in Table 1.

TABLE 1
DNA Collection and Protein Rejection.

| | | | | | | | Number | |
|---|---|---|---|---|---|---|---|---|
| | Input DNA | Output DNA | | Input Protein | Output Protein | Protein | of SCODA | Number |
| | Concentration | Concentration | Collection | Concentration | concentration | Rejection | Focusing | of Wash |
| Sample Type | (μg/mL) | (μg/mL) | Ratio | (μg/mL) | (μg/mL) | Ratio | Cycles | Steps |
| Human serum | 0.420 +/− 0.08 | 0.303 +/− 0.03 | 72.1% | 55000 +/− 500 | <25 | >2200 | 300 | 15 |
| spiked with | | | | | | | | |
| λDNA | | | | | | | | |
| Cell lysate | 0.158 +/− 0.02 | 0.129 +/− 0.01 | 81.6% | 1400 +/− 80 | <25 | >56 | | |
| (1:10 diluted) | | | | | | | | |
| Blood lysate | 0.528 +/− 0.03 | 0.391 +/− 0.04 | 74.0% | 3900 +/− 190 | <25 | >156 | | |
| (1:20 diluted) | | | | | | | | |

The sieve capacity to obtain amplifiable DNA from a crude sample has been tested on the lysates of cultured cells and whole rabbit blood using real-time quantitative PCR (RT-qPCR).

FIGS. 5A-5B show the amplification curves of a housekeeping gene (GAPDH) in SCODA-purified DNA samples from cell and blood lysates, respectively. The lysates had to be diluted (the cell lysate by 10-fold and the whole blood lysate by 20-fold) with deionized water to bring the ionic strength and conductivity levels closer to those of the working buffer to facilitate electrophoretic injection into the sieve. The cycle threshold values were determined to be 28.7±0.14 and 25.05±0.1 (n=3), which correspond to 3±0.3×10³ and 4.7±0.3×10⁴ initial copies, respectively, based on standards (FIG. 14).

Accelerated electrophoretic focusing and purification of DNA is provided, reducing the time required from hours to minutes by performing SCODA on a microfabricated sieve instead of a polymer gel, under an increased field strength. With an appropriate electrophoretic protocol, the sieve can deliver amplifiable nucleic acid from a crude lysate. The sieve is amenable to further integration with nucleic acid amplification and detection modules for a sample-in answer-out point-of-care diagnostic system.

### EXAMPLE 2

An embodiment of the present application involves artificial gels with a plurality of micropillars arranged in the form of an array on a microchip. The microchip may consist of a purification chamber surrounded by injection regions for sample loading (FIG. 6). An elastomer (polydimethylsiloxane, PDMS) slab with fluidic and electrical vias may be used to enclose the purification chamber and injection regions. The micropillars may have the layout of a circle, square, rectangle, cross, and cross with slits (FIG. 9). The method uses a pre-programmed sequence of voltage waveforms delivered to the electrodes placed around the purification chamber inside the reservoirs labelled as A, B, C, and D in FIG. 6. An example of this sequence of voltage pulses is also listed in FIG. 6. This sequence establishes a primary electric field that drives molecules within the artificial gel or sieving layer, as well as a secondary electric field that alters the electrophoretic mobility of molecules within the layer. Nucleic acids may be rapidly driven to a particular zone in the purification chamber within a few minutes, where they are converged and spatially separated from most unwanted molecules and contaminants especially proteins (FIGS. 10-12). The enriched and purified nucleic acids may be collected for further analysis or may undergo various on-chip amplification techniques, such as PCR or LAMP for the qualitative and quantitative analysis of target nucleic acids.

### Fabrication

Artificial gels may be fabricated by using either of the two alternative processes described below. The fabrication may use a silicon wafer with a thickness of 525 μm. FIGS. 7 and 8 illustrate the fabrication processes. Process I (FIG. 7): silicon micropillars may be formed by deep reactive ion etching silicon after a lithographic patterning. The height of the micropillars may be controlled by etching duration. Then, a thin-film oxide with a thickness of 500 nm may be placed on the micropillars through wet oxidation. The pillar-to-pillar spacing is also controlled through the oxidation process. This oxide layer serves as a dielectric barrier for electrical passivation and a layer to facilitate bonding the silicon substrate with an elastomer polydimethylsiloxane (PDMS) slab to enclose the gel. Process II (FIG. 8): micropillars are built into a thick layer of oxide deposited onto the silicon substrate. Then the micro-pillars may be formed by etching the oxide layer after a lithographic patterning. Finally, a PDMS slab with fluidic and electrical vias may be placed and secured over the micropillars after oxygen plasma surface activation.

The embodiments of the subject invention replace the conventional polymer gels, which are typically used in electrophoresis, with artificial gels for rapid electrophoretic focusing and purification of nucleic acids from other molecules and contaminants. Polymer gels are sensitive to temperature (Joule heating), a feature that inhibits their use with high voltage to perform rapid electrophoretic purification of nucleic acids. Thus, electrophoretic purification of nucleic acids inside polymer gels may take an extended period of time. Moreover, because of this limitation, polymer gels often require a cooling system during use which increases the complexity of the required instrument and often leads to other issues due to potential temperature nonuniformities. Artificial gels with the ability to sustain high temperature and high voltages cut this purification time to minute(s). Further, artificial gels are planar structures with limited depth or height (a relatively thin layer) which limits the generation of Joule heat while enhancing the dissipation of heat once generated due to large surface-area-to-volume ratio. Consequently, they can be used with relatively large voltages without the concern of joule heating and associated problems.

### EXAMPLE 3

Methicillin-resistant Staphylococcus aureus (MRSA) is a life-threatening pathogen with strong antibiotic-resistance that can cause severe bloodstream infection and lead to outbreaks in community [30]. Thus, rapid MRSA detection with high accuracy is crucial for early diagnosis before further exacerbation of infection and disease. However, conventional detection methods based on microbial culture are usually time-consuming, labor-intensive and costly. Here, we present a sample-to-answer integrated microfluidic system for rapid MRSA detection [31]. This system takes serum as input, then releases nucleic acids from MRSA bacteria by on-chip lysis, followed by electrophoretic purification, finally the nucleic acids detection is realized by colorimetric loop-mediated isothermal amplification (LAMP).

FIG. 15A shows the exploded schematic view of the microfluidic system, which is composed of a heater, a silicon chip, polydimethylsiloxane slabs with fluidic and electrical access holes, and four platinum electrodes to apply the electric field. FIG. 15B shows the workflow for pathogen detection, starting from adding serum sample (spiked with MRSA, 100 CFU/reaction) to the input reservoir, on-chip lysis and DNA purification, then colorimetric results were obtained after on-chip LAMP. FIG. 15C shows the nucleic acids purification steps by synchronous coefficient of drag alteration (SCODA) on the microfluidic chip [32]. This electrophoretic purification process isolates DNA from MRSA lysate, washes out contaminants, then the purified DNAs are transported by electrophoresis to the LAMP reaction reservoir for colorimetric detection.

The colorimetric results were quantified in real-time using the red-green-blue data obtained from two color sensors that respectively read from positive and negative controls. FIG. 15D shows both a side and top-down view of the microfluidic chip. The side view highlights post-LAMP reservoirs containing a positive sample and a no-template control (negative sample), while the top-down view depicts the chip setup configured for real-time monitoring of colorimetric LAMP reactions. MRSA-positive samples exhibit a green color, while MRSA-negative (no-template control) samples appear brown because the ion-indicator dye transforms color by the formation of non-soluble by-product that decreases ion concentration during LAMP. The real-time color transition over the 15-min LAMP reaction is plotted in FIG. 15E, exhibiting distinct color difference between MRSA-positive and negative-control samples.

While the SCODA and method of use thereof is used to detect MRSA in a sample in the embodiment described above, a person of ordinary skill in the art would readily appreciate that the SCODA and methods described herein could be used to detect nucleic acids in any type of sample. In certain embodiments, the sample comprises or is derived from a biological sample, such as a blood sample, a mucus sample, a lung lavage sample, a urine sample, a fecal sample, a skin sample, a hair sample, a semen sample, a vaginal sample, an amniotic sample, a cell sample, or a cell lysate sample; or an environmental sample, such as a soil sample, a soil sample, a water sample, an air sample, a meat sample, a vegetable sample or a fruit sample.

In certain embodiments, the SCODA and method of use thereof are used to identify the presence of nucleic acids from an infectious agent in the sample, such as a bacteria, virus, fungi, mycoplasm or prion. In certain embodiments, the bacteria comprises an antibiotic resistant bacteria. Exemplary antibiotic resistant bacteria include, but are not limited to, methicillin-resistant Staphylococcus aureus (MRSA), vancomycin-resistant Enterococci (VRE), carbapenem-resistant Enterobacteriaceae (CRE), antibiotic-resistant Streptococcus pneumoniae, daptomycin-resistant Staphylococcus aureus (DRSA), linezolid-resistant Staphylococcus aureus (LRSA), antibiotic-resistant Mycobacterium tuberculosis (MDR-TB), fluoroquinolone-resistant salmonella and shigella, antibiotic-resistant Pseudomonas aeruginosa, carbapenam-resistant Acinetobacter, antibiotic-resistant Neisseria gonorrhoeae, and antibiotic-resistant Candida auris.

In certain embodiments, the SCODA and method of use thereof are used in a method of diagnosis of a hereditary disease or cancer, wherein the hereditary disease or cancer is caused by one or more DNA mutations, e.g., triplet base expansions, base substitution mutations, deletion mutations, addition mutations, nonsense mutations, premature stop codons, chromosomal deletions, chromosomal duplications, aneuploidy, partial aneuploidy or monosomy.

### EXEMPLARY EMBODIMENTS

Embodiment 1. A synchronous coefficient of drag alteration (SCODA) method for concentrating and purifying nucleic acids from a sample containing unwanted molecules and contaminants, comprising:
providing a layer of artificial gel or sieve filled with liquid and into which molecules including nucleic acids are introduced from a sample liquid by applying a steady electric field across a boundary between the artificial gel layer and the sample liquid containing the molecules including nucleic acids; and
providing a plurality of electrodes spaced apart around a circumference of the artificial gel layer and connected to at least one external power supply for delivering a pre-programmed sequence of voltage waveforms to establish a primary electric field driving molecules within the artificial gel layer and a secondary electric field altering electrophoretic mobility of molecules within the artificial gel layer;
wherein the plurality of electrodes is configured to generate a pre-programmed sequence of voltage waveforms such that the nucleic acids alone are driven to a certain zone in the artificial gel layer where they are converged and spatially separated from the unwanted molecules and the contaminants to be relatively purified and enriched.

Embodiment 2. The method according to embodiment 1, wherein the primary electric field and secondary electric field are established in the artificial gel layer concurrently.

Embodiment 3. The method according to embodiment 1, wherein the sample introduction and nucleic acid enrichment and purification are performed concurrently.

Embodiment 4. The method according to embodiment 1, wherein the artificial sieve layer is made of silicon-based, silica-based, or polymer-based insulating or dielectric materials through cleanroom processes involving lithography.

Embodiment 5. The method according to embodiment 1, wherein the artificial sieve layer is made of assembled layer(s) of colloids, bubbles or foam, droplets, nanowires, or nanotubes.

Embodiment 6. The method according to embodiment 1, wherein the artificial sieve layer is configured to sustain a high voltage exceeding 100 V applied across the artificial sieve and sustain a temperature elevated above room temperature due to Joule heating.

Embodiment 7. The method according to embodiment 1, further comprising collecting the enriched and purified nucleic acids from the artificial sieve layer for downstream use.

Embodiment 8. The method according to embodiment 1, further comprising applying polymerase chain reaction or loop-mediated isothermal amplification to the enriched and purified nucleic acids without collecting the nucleic acids from the artificial gel layer.

Embodiment 9. The method according to embodiment 8, wherein the amplified target sequence or sequences are detected in real time or following the amplification process, without collecting the amplified products from the artificial gel layer.

Embodiment 10. The method according to embodiment 8, wherein a reaction temperature is controlled by external means and hardware interfacing with the artificial gel layer or a fluidic reservoir associated with the artificial gel layer.

Embodiment 11. The method according to embodiment 9, wherein the detection of the amplified products is achieved by electrical or optical hardware interfacing with the artificial gel layer or a fluidic reservoir associated with the artificial gel layer.

Embodiment 12. A synchronous coefficient of drag alteration (SCODA) system for concentrating and purifying nucleic acids from a sample containing unwanted molecules and contaminants, comprising:
a layer of artificial gel or sieve filled with liquid and configured to receive molecules including nucleic acids that are introduced from a sample liquid by applying a steady electric field across a boundary between the artificial gel layer and the sample liquid containing the molecules including nucleic acids; and
a plurality of electrodes spaced apart around a circumference of the artificial gel layer and configured to connect to at least one external power supply for delivering a pre-programmed sequence of voltage waveforms to establish a primary electric field driving molecules within the artificial gel layer and a secondary electric field altering electrophoretic mobility of molecules within the artificial gel layer;
wherein the plurality of electrodes are configured to generate a pre-programmed sequence of voltage waveforms such that the nucleic acids alone are driven to a certain zone in the artificial gel layer where they are converged and spatially separated from the unwanted molecules and the contaminants to be relatively purified and enriched.

Embodiment 13. The system according to embodiment 12, wherein the primary electric field and secondary electric field are established in the artificial gel layer concurrently.

Embodiment 14. The system according to embodiment 12, wherein the sample introduction and nucleic acid enrichment and purification are performed concurrently.

Embodiment 15. The system according to embodiment 12, wherein the artificial sieve layer is made of silicon-based, silica-based, or polymer-based insulating or dielectric materials through cleanroom processes involving lithography.

Embodiment 16. The system according to embodiment 12, wherein the artificial sieve layer is made of assembled layer(s) of colloids, bubbles or foam, droplets, nanowires, or nanotubes.

Embodiment 17. The system according to embodiment 12, wherein the artificial sieve layer is configured to sustain a high voltage exceeding 100 V applied across the artificial sieve and sustain a temperature elevated above room temperature due to Joule heating.

Embodiment 18. The system according to embodiment 12, wherein the nucleic acids enriched and purified from the unwanted molecules and the contaminants are collected from the artificial sieve layer for downstream use.

Embodiment 19. The system according to embodiment 12, wherein the enriched and purified nucleic acids undergo polymerase chain reaction or loop-mediated isothermal amplification separate from the unwanted molecules and contaminants, without collecting the nucleic acids from the artificial gel layer.

Embodiment 20. The system according to embodiment 19, wherein the amplified target sequence or sequences are detected in real time or following the amplification process, without collecting the amplified products from the artificial gel layer.

Embodiment 21. The method according to embodiment 1, wherein the enriched and purified nucleic acids are collected by a fluidic reservoir in connection with the artificial gel layer or the sieve via electrophoretic transport.

Embodiment 22. The method according to embodiment 21, wherein the enriched and purified nucleic acids are transported under a steady bias to the reservoir after being concentrated and purified in the artificial gel or the sieve through the SCODA focusing.

Embodiment 23. The method according to embodiment 21, further comprising applying polymerase chain reaction or loop-mediated isothermal amplification to the enriched and purified nucleic acids in the fluidic reservoir connected to the artificial gel layer after transporting the enriched and purified nucleic acids to the reservoir.

Embodiment 24. The method according to embodiment 1, further comprising a step of electrophoretic washing the artificial gel or sieve to gradually wash the contaminants out of the artificial gel or sieve.

Embodiment 25. The method according to embodiment 24, wherein the step of electrophoretic washing comprises applying a pulse of 180 Vₚ for 1 second to a reservoir after the SCODA focusing cycle.

Embodiment 26. The method according to embodiment 25, wherein the SCODA focusing cycle comprises a plurality of rounds of activation of the plurality of electrodes in sequence.

Embodiment 27. The method according to embodiment 1, wherein following electrophoretic washing steps are performed between the electrophoretic focusing cycles:
conducting a first electrophoretic washing of the enriched and purified nucleic acids;
conducting another electrophoretic focusing; and
conducting a second electrophoretic washing of the nucleic acids.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. LA Christel K Peterson W McMillan MA Northrup Rapid automated nucleic acid probe assays using silicon microstructures for NA concentration J Biomech Eng 1999 121 22.
2. NC Cady S Stelick CA Batt Nucleic acid purification using microfabricated silicon structures Biosens Bioelectron 2003 19 59.
3. L Yobas HM Ji W Hui Y Chen TM Lim CK Heng DL Kwong Nucleic acid extraction, amplification, and detection on Si-based microfluidic platforms IEEE J Solid-State Circuits 2007 42 1803.
4. WC Hui L Yobas VD Samper CK Heng S Liw HM Ji Y Chen C Lin J Li TM Lim Microfluidic systems for extracting nucleic acids for DNA and RNA analysis Sens and Actuat A 2007 133 335.
5. H Tian AFR Huhmer JP Landers Evaluation of silica resins for direct and efficient extraction of DNA from complex biological matrices in a miniaturized format Anal Biochem 2000 283 175.
6. KA Wolfe MC Breadmore JP Ferrance ME Power JF Conroy PM Norris JP Landers Toward a microchipbased solid-phase extraction method for isolation of nucleic acids Electrophoresis 2002 23 727.
7. CW Price DC Leslie JP Landers Nucleic acid extraction techniques and application to the microchip Lab Chip 2009 9 2484.
8. T Nakagawa T Tanaka D Niwa T Osaka H Takeyama T Matsunaga Fabrication of amino silane-coated microchip for DNA extraction from whole blood J Biotechnol 2005 116 105.
9. W Cao CJ Easley JP Ferrance JP Landers Chitosan as a Polymer for pH-Induced DNA Capture in a Totally Aqueous System Anal Chem 2006 78 27222.
10. Y Xu B Vaidya AB Patel SM Ford RL McCarley SA Soper Solid-Phase Reversible Immobilization in Microfluidic Chips for the Purification of Dye-Labeled DNA Sequencing Fragments Anal Chem 2003 75 2975.
11. J Rajendhran P Gunasekaran Strategies for accessing soil metagenome for desired applications Biotechnol Adv 2008 26 576.
12. A Marziali J Pel D Bizzotto LA Whitehead Novel electrophoresis mechanism based on synchronous alternating drag perturbation Electrophoresis 2005 26 82.
13. J Pel D Broemeling L Mai HL Poon G Tropini RL Warren RA Holt A Marziali Nonlinear electrophoretic response yields a unique parameter for separation of biomolecules Proc Natl Acad Sci USA 2009 106 14796.
14. DJ Broemeling J Pel DC Gunn L Mai JD Thompson H Poon A Marziali An instrument for automated purification of nucleic acids from contaminated forensic samples JALA 2008 13 40.
15. S Schmedes P Marshall JL King B Budowle Effective removal of co-purified inhibitors from extracted DNA samples using synchronous coefficient of drag alteration (SCODA) technology Int J Legal Med 2013 127 749.
16. VN Kondratova OI Serd'uk VP Shelepov AV Lichtenstein Concentration and isolation of DNA from biological fluids by agarose gel isotachophoresis Biotechniques 2005 39 695.
17. A Rogacs LA Marshall JG Santiago Purification of nucleic acids using isotachophoresis J Chromatogr A 2014 1335 105.
18. A Persat LA Marshall JG Santiago Purification of Nucleic Acids from Whole Blood Using Isotachophoresis Anal Chem 2009 81 9507.
19. M. Bercovici GV Kaigala KE Mach CM Han JC Liao JG Santiago Rapid Detection of Urinary Tract Infections Using Isotachophoresis and Molecular Beacons Anal Chem 2011 83 4110.
20. A Rogacs Y Qu JG Santiago Bacterial RNA Extraction and Purification from Whole Human Blood Using Isotachophoresis Anal Chem 2012 84 5858.
21. RB Schoch M Ronaghi JG Santiago Rapid and selective extraction, isolation, preconcentration, and quantitation of small RNAs from cell lysate using on-chip isotachophoresis Lab Chip 2009 9 2145.
22. YC Wang AL Stevens J Han Million-fold Preconcentration of Proteins and Peptides by Nanofluidic Filter Anal Chem 2005 77 4293.
23. SJ Kim SH Ko KH Kang J Han Direct seawater desalination by ion concentration polarization Nat Nanotechnology 2010 5 297.
24. LF Cheow J Han Continuous Signal Enhancement for Sensitive Aptamer Affinity Probe Electrophoresis Assay Using Electrokinetic Concentration Anal Chem 2011 83 7086
25. W Ouyang Z Li J Han Pressure-Modulated Selective Electrokinetic Trapping for Direct Enrichment Purification and Detection of Nucleic Acids in Human Serum Anal Chem 2018 90 11366.
26. P Agrawal Z Bognár KD Dorfman Entropic trap purification of long DNA Lab Chip 2018 18 955.
27. J Han HG Craighead Separation of long DNA molecules in a microfabricated entropic trap array Science 2000 288 1026.
28. C Heller T Duke JL Viovy Electrophoretic mobility of DNA in gels. II. Systematic experimental study in agarose gels Biopolymers 1994 34 249.
29. J Rousseau G Drouin GW Slater Gel electrophoretic mobility of single-stranded DNA Electrophoresis 2000 21 1464.30. H Humphreys Can we do better in controlling and preventing methicillin-resistant Staphylococcus aureus (MRSA) in the intensive care unit (ICU)? Eur J Clin Microbiol Infect Dis 2008 27 409.
31. YC Lee Y Bu S Ni Y Liu A Hu L Yobas A compact sample-to-answer system for rapid MRSA detection in serum based on reagent-free electrophoretic purification of nucleic acids and colorimetric LAMP Lab Chip 2025 (DOI: 10.1039/d5lc00152h).
32. Y Bu S Ni L Yobas Accelerated Electrophoretic Focusing and Purification of DNA Based on Synchronous Coefficient of Drag Alteration Analytical Chemistry 2023 95 16453.

We claim:

1. A synchronous coefficient of drag alteration (SCODA) method for concentrating and purifying nucleic acids from a sample containing unwanted molecules and contaminants, comprising:
providing a layer of artificial gel or sieve filled with liquid and into which molecules including nucleic acids are introduced from a sample liquid by applying a steady electric field across a boundary between the artificial gel layer and the sample liquid containing the molecules including nucleic acids; and
providing a plurality of electrodes spaced apart around a circumference of the artificial gel layer and connected to at least one external power supply for delivering a pre-programmed sequence of voltage waveforms to establish a primary electric field driving molecules within the artificial gel layer and a secondary electric field altering electrophoretic mobility of molecules within the artificial gel layer;
wherein the plurality of electrodes is configured to generate a pre-programmed sequence of voltage waveforms such that the nucleic acids alone are driven to a certain zone in the artificial gel layer where they are converged and spatially separated from the unwanted molecules and the contaminants to be relatively purified and enriched.

2. The method according to claim 1, wherein the primary electric field and secondary electric field are established in the artificial gel layer concurrently.

3. The method according to claim 1, wherein the sample introduction and nucleic acid enrichment and purification are performed concurrently.

4. The method according to claim 1, wherein the artificial sieve layer is made of silicon-based, silica-based, or polymer-based insulating or dielectric materials through cleanroom processes involving lithography.

5. The method according to claim 1, wherein the artificial sieve layer is made of assembled layer(s) of colloids, bubbles or foam, droplets, nanowires, or nanotubes.

6. The method according to claim 1, wherein the artificial sieve layer is configured to sustain a voltage exceeding 100 V applied across the artificial sieve and sustain a temperature elevated above room temperature due to Joule heating.

7. The method according to claim 1, further comprising collecting the enriched and purified nucleic acids from the artificial sieve layer for downstream use.

8. The method according to claim 1, further comprising applying polymerase chain reaction or loop-mediated isothermal amplification to the enriched and purified nucleic acids without collecting the nucleic acids from the artificial gel layer.

9. The method according to claim 8, wherein the amplified target sequence or sequences are detected in real time or following the amplification process, without collecting the amplified products from the artificial gel layer.

10. The method according to claim 8, wherein a reaction temperature is controlled by external means and hardware interfacing with the artificial gel layer or a fluidic reservoir associated with the artificial gel layer.

11. The method according to claim 9, wherein the detection of the amplified products is achieved by electrical or optical hardware interfacing with the artificial gel layer or a fluidic reservoir associated with the artificial gel layer.

12. A synchronous coefficient of drag alteration (SCODA) system for concentrating and purifying nucleic acids from a sample containing unwanted molecules and contaminants, comprising:
a layer of artificial gel or sieve filled with liquid and configured to receive molecules including nucleic acids that are introduced from a sample liquid by applying a steady electric field across a boundary between the artificial gel layer and the sample liquid containing the molecules including nucleic acids; and
a plurality of electrodes spaced apart around a circumference of the artificial gel layer and configured to connect to at least one external power supply for delivering a pre-programmed sequence of voltage waveforms to establish a primary electric field driving molecules within the artificial gel layer and a secondary electric field altering electrophoretic mobility of molecules within the artificial gel layer;
wherein the plurality of electrodes are configured to generate a pre-programmed sequence of voltage waveforms such that the nucleic acids alone are driven to a certain zone in the artificial gel layer where they are converged and spatially separated from the unwanted molecules and the contaminants to be relatively purified and enriched.

13. The system according to claim 12, wherein the primary electric field and secondary electric field are established in the artificial gel layer concurrently.

14. The system according to claim 12, wherein the sample introduction and nucleic acid enrichment and purification are performed concurrently.

15. The system according to claim 12, wherein the artificial sieve layer is made of silicon-based, silica-based, or polymer-based insulating or dielectric materials through cleanroom processes involving lithography.

16. The system according to claim 12, wherein the artificial sieve layer is made of assembled layer(s) of colloids, bubbles or foam, droplets, nanowires, or nanotubes.

17. The system according to claim 12, wherein the artificial sieve layer is configured to sustain a voltage exceeding 100 V applied across the artificial sieve and sustain a temperature elevated above room temperature due to Joule heating

18. The system according to claim 12, wherein the nucleic acids enriched and purified from the unwanted molecules and the contaminants are collected from the artificial sieve layer for downstream use.

19. The system according to claim 12, wherein the enriched and purified nucleic acids undergo polymerase chain reaction or loop-mediated isothermal amplification separate from the unwanted molecules and contaminants, without collecting the nucleic acids from the artificial gel layer.

20. The system according to claim 19, wherein the amplified target sequence or sequences are detected in real time or following the amplification process, without collecting the amplified products from the artificial gel layer.

21. The method according to claim 1, wherein the enriched and purified nucleic acids are collected by a fluidic reservoir in connection with the artificial gel layer or the sieve via electrophoretic transport.

22. The method according to claim 21, wherein the enriched and purified nucleic acids are transported under a steady bias to the reservoir after being concentrated and purified in the artificial gel or the sieve through the SCODA focusing.

23. The method according to claim 21, further comprising applying polymerase chain reaction or loop-mediated isothermal amplification to the enriched and purified nucleic acids in the fluidic reservoir connected to the artificial gel layer after transporting the enriched and purified nucleic acids to the reservoir.

24. The method according to claim 1, further comprising a step of electrophoretic washing the artificial gel or sieve to gradually wash the contaminants out of the artificial gel or sieve.

25. The method according to claim 24, wherein the step of electrophoretic washing comprises applying a pulse of 180 Vₚ for 1 second to a reservoir after the SCODA focusing cycle.

26. The method according to claim 25, wherein the SCODA focusing cycle comprises a plurality of rounds of activation of the plurality of electrodes in sequence.

27. The method according to claim 1, wherein the following electrophoretic washing steps are performed between the electrophoretic focusing cycles:
conducting a first electrophoretic washing of the enriched and purified nucleic acids;
conducting another electrophoretic focusing; and
conducting a second electrophoretic washing of the nucleic acids.
